(19) United States
(12) Patent Application Publication
(10) Pub. No.: US 2019/0117239 A1
(43) Pub. Date: Apr. 25, 2019

(54) Patient-Specific Surgical Tool Guide

(71) Applicant: Nikhil N. Verma, Chicago, IL (US)

(72) Inventor: Nikhil N. Verma, Chicago, IL (US)

(21) Appl. No.: 16/171,253

(22) Filed: Oct. 25, 2018

Related U.S. Application Data
(60) Provisional application No. 62/577,044, filed on Oct. 25, 2017.

(51) Int. Cl. A61B 17/17; B33Y 50/00; B33Y 80/00

(52) U.S. Cl. CPC A61B 17/1778; B33Y 80/00; B33Y 50/00

(57) ABSTRACT

A method for creating a custom-fit guide for us in precision application such as orthopedic surgeries. Scanning technology is used to non-invasively create a 3D object of a targeted area. A guide is virtually added to the targeted area and later isolated and used as input to a 3D printer to create a physical guide for use in the procedure.

### BACKGROUND OF THE INVENTION

Orthopedic surgery often entails the precise placement of surgical implants, such as pins and screws or other implants, into bone tissue. The irregular surfaces of bones and the complicated degeneration that often occurs with various diseases states make the placement of these implants, or the precise placement of pilot holes for receiving the implants, difficult.

By way of non-limiting example, total shoulder and reverse shoulder arthroplasty is a common means to treat glenohumeral arthritis, primary or secondary after failed conservative management. More than 50,000 people undergo a total shoulder operation each year in the United States. In a shoulder joint replacement, the boney aspects of shoulder joint consists of the glenoid and the humeral head, with the glenoid being the socket and the humeral head making up the ball. Each of these bony surfaces are cut away and replaced with an artificial component. To recreate the correct anatomic alignment and position of the joint, the surgeon must carefully plan how much bone to remove from the glenoid and humeral head. Additionally, it is imperative that the surgeon determines the correct angle and orientation for component insertion, preparing the bone such that the artificial components can be securely fixed to the native bone in the correct orientation. This is often difficult to do as the disease process of arthritis or other shoulder pathology involves the wearing-away of bone and the development of new bone spurs and boney prominences in non-native locations. This process changes the orientation and shape of the glenoid face, making it difficult to determine how bone resection/preparation should occur to recreate a healthy, normal anatomical joint once the artificial components are in place.

Surgeons often use computer tomography scans (CT scans) to visualize the patient's anatomy and plan the precise angles at which to remove the bone. However, when it comes to performing the surgery these plans are often carried out freehanded by the surgeon leaving many sources for error.

There is thus a need for a method and device that is customizable to an individual patient and ensure the precise placement of surgical implants and bone reshaping efforts.

### SUMMARY OF THE INVENTION

The above-mentioned need is met by a novel technique for creating a custom guide useable in various surgical procedures. Using shoulder surgery as an example, this new technique is used to create a customized drill guide to assist in the implementation of the glenoid component of the total shoulder arthroplasty that is specifically designed based on patient-specific anatomical shoulder morphology.

One aspect of the invention involves acquiring a CT scan of the patient's anatomy, rendering the CT data of the scapula and glenoid into a three-dimensional (3D) object, calculating what the normal glenoid-version and surface-plane should be, based off population based data and the type of implant being used. The CT data is then used to develop a 3D drill guide for the surgical implementation, which is then 3D printed and sterilized. The goal of the 3D printed drill guide is to help place a surgical guide pin in the correct orientation on the face of the glenoid and at an angle that is normalized for anatomic glenoid version in two planes. The 3D guide is placed on the patient's arthritic glenoid and a guide pin is inserted into the glenoid with the use of the guide. The guide is then removed from the glenoid, leaving the drill pin securely fixed in the glenoid bone. Once the appropriate reference for normal version is achieved using the guide pin, glenoid preparation tools such as cannulated reamer is then placed over the guide pin allowing the guide pin to orient the reamer correctly to prepare the glenoid as per pre-operative planning leaving a bone surface that when artificial glenoid component is placed, the original, healthy, non-arthritic ideal version of the glenoid is recreated.

Another aspect of the invention pertains to a method of creating a patient-specific surgical tool guide comprising: scanning a target location on a patient for which a tool guide is desired; using data from said scanning to create a virtual solid object; identifying a surface on said image with which to mate said tool guide; creating a virtual tool guide against said surface; and creating a physical tool guide from said virtual tool guide.

In one aspect, scanning the target location on a patient for which a tool guide is desired comprises scanning the patient using a CT imager.

In another aspect, scanning to create a virtual solid object comprises: segmenting areas of interest of the target location; subtracting areas not of interest from the data; rendering the data into a 3D object; saving said 3D object as a stereolithography (STL) file; using a 3D modeling program to convert said STL file into said virtual solid object.

In another aspect segmenting areas of interest of the target location comprises filling in gaps in said data, said gaps occurring between scans of said CT scanning.

Yet another aspect involves rotating said virtual object to achieve an orthogonal view of said surface.

Another aspect includes establishing a center of said surface on said orthogonal view. This may include placing two perpendicular intersecting lines on said surface, each line located to span a widest and a tallest dimension of said surface, respectively. This aspect may further include creating a third line through an intersection of said two perpendicular lines, said third line representing an axis along which a tool will be inserted into the guide. The third line may be perpendicular to said two intersecting lines.

In another aspect, creating a physical tool guide comprises 3D printing an object using from a file of the virtual tool guide.

Yet another aspect of the invention is a method of using a tool to modify a bone of a patient comprising: creating a tool guide having a mating surface that mates with a target surface of a bone of a patient, said mating surface is created using from a virtual model of said target surface; inserting a tool through an opening in said tool guide that aligns said tool with said target surface; and, using said tool to modify said bone.

The step of creating a tool guide may involve: scanning a target location containing the target surface; creating said virtual model; identifying said target surface representation on said virtual solid object; creating a virtual tool guide against said surface; creating said tool guide from said virtual tool guide.

Another aspect of the invention is a tool guide for use in modifying a bone of a patient comprising: a surface shaped to mate with a target surface of a bone of a patient; and an opening sized to accommodate a tool and leading to said target surface.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 is wireframe representation of a scapula created as a step of an embodiment of a method of the invention;

FIG. 2a is a top view of the scapula of FIG. 1 rendered as a solid object created as a step of an embodiment of a method of the invention;

FIG. 2b is a perspective view of the scapula of FIG. 1 rendered as a solid object created as a step of an embodiment of a method of the invention;

FIG. 2c is a front view of the scapula of FIG. 1 rendered as a solid object created as a step of an embodiment of a method of the invention;

FIG. 2d is a right view of the scapula of FIG. 1 rendered as a solid object created as a step of an embodiment of a method of the invention;

FIG. 3a is an en face view of the glenoid of the scapula of FIG. 1 rendered as a solid object created as a step of an embodiment of a method of the invention;

FIG. 3b is the view of FIG. 3a with a center identified according to a step of an embodiment of a method of the invention;

FIG. 4a is a perspective view of the scapula of FIG. 2 including a line drawn from the trigonum of the medial border of the scapula to the intersection of the lines drawn in FIG. 3b, according to a step of an embodiment of a method of the invention;

FIG. 4b is a perspective view of the scapula of FIG. 4a showing the glenoid face;

FIG. 5 is a perspective view of the scapula of FIG. 2 with the glenoid surface traced and marked according to a step of an embodiment of a method of the invention;

FIG. 6a is a top view of the scapula rendering of FIG. 2 with a virtual drill guide created on the glenoid face according to a step of an embodiment of a method of the invention;

FIG. 6b is a perspective view of the scapula rendering of FIG. 2 with a virtual drill guide created on the glenoid face according to a step of an embodiment of a method of the invention;

FIG. 6c is a front view of the scapula rendering of FIG. 2 with a virtual drill guide created on the glenoid face according to a step of an embodiment of a method of the invention;

FIG. 6d is a right view of the scapula rendering of FIG. 2 with a virtual drill guide created on the glenoid face according to a step of an embodiment of a method of the invention;

FIG. 7a is a top view of the drill guide of FIG. 6a;

FIG. 7b is a perspective view of the drill guide of FIG. 6b;

FIG. 7c is a front view of the drill guide of FIG. 6c;

FIG. 7d is a right view of the drill guide of FIG. 6d;

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

For sake of clarity, the non-limiting example of a shoulder is used throughout the specification to illustrate an embodiment of a method of the invention. One skilled in the art will realize that the invention can be used on different parts of the body, especially in the field of orthopedic or reconstructive surgery. The invention has applicability outside of the surgical field as well.

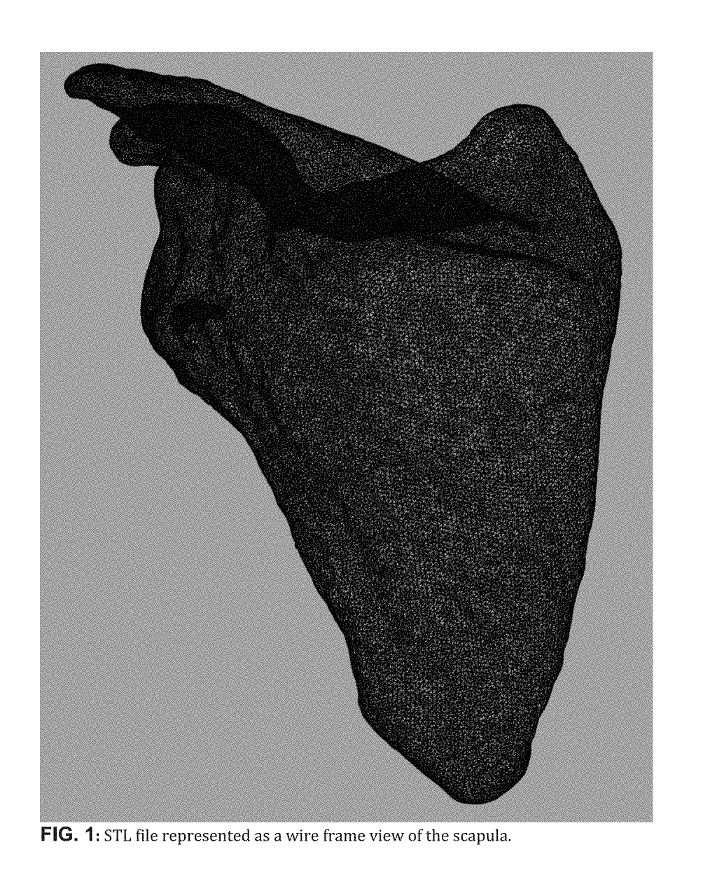

An embodiment of the invention begins with a pre-operative scan of the shoulder to create a digital image of the shoulder. An example of a scanning technology is CT. This scan can then be imported into open sourced or commercial software package that allows the user to segment the CT into a 3D object. This requires segmentation of the bones of interest (glenoid and scapula) subtracting the remainder of the joint. These highlighted portions of the scan can then be rendered into a 3D object with the software package filling in the gaps between each image slice to make a solid object. This object is then saved as a stereolithography (STL) file, which is a file format that saves the data of the 3D object as a wire frame of the surface of the object. For this technique, this is done to create a STL file of the scapula with particular focus on carefully segmenting the glenoid surface of the scapula. FIG. 1 shows a wire frame representation of the scanned glenoid and scapula.

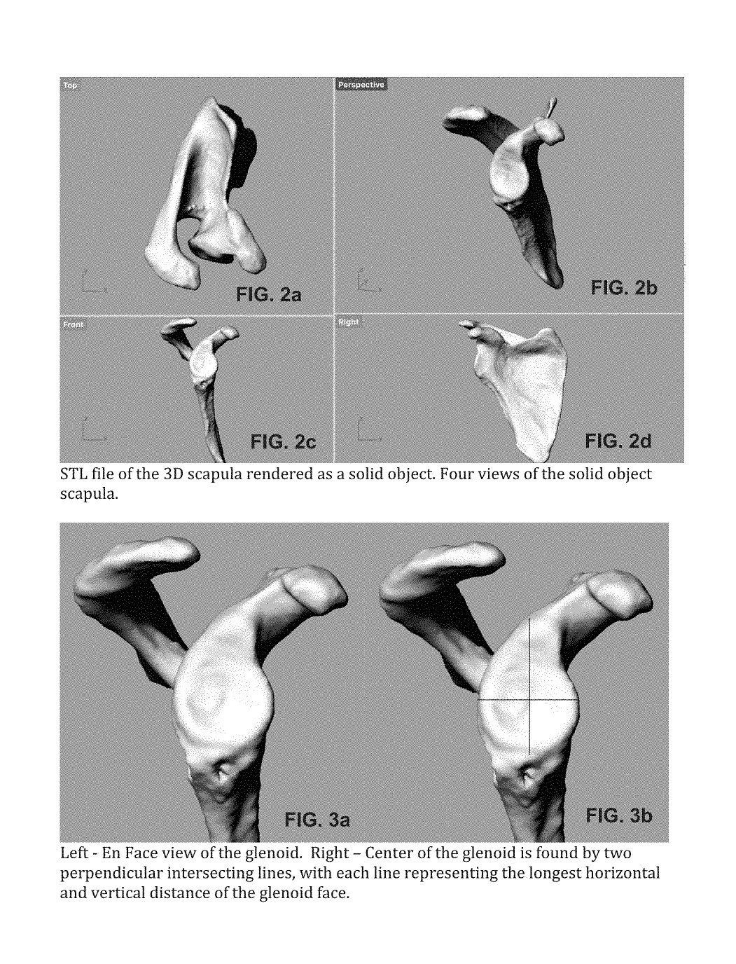

Referring now to FIGS. 2a-2d, once the STL file of the scapula is created, the file is imported into a 3D modeling software. This software is then used to turn the STL file into a virtual solid object.

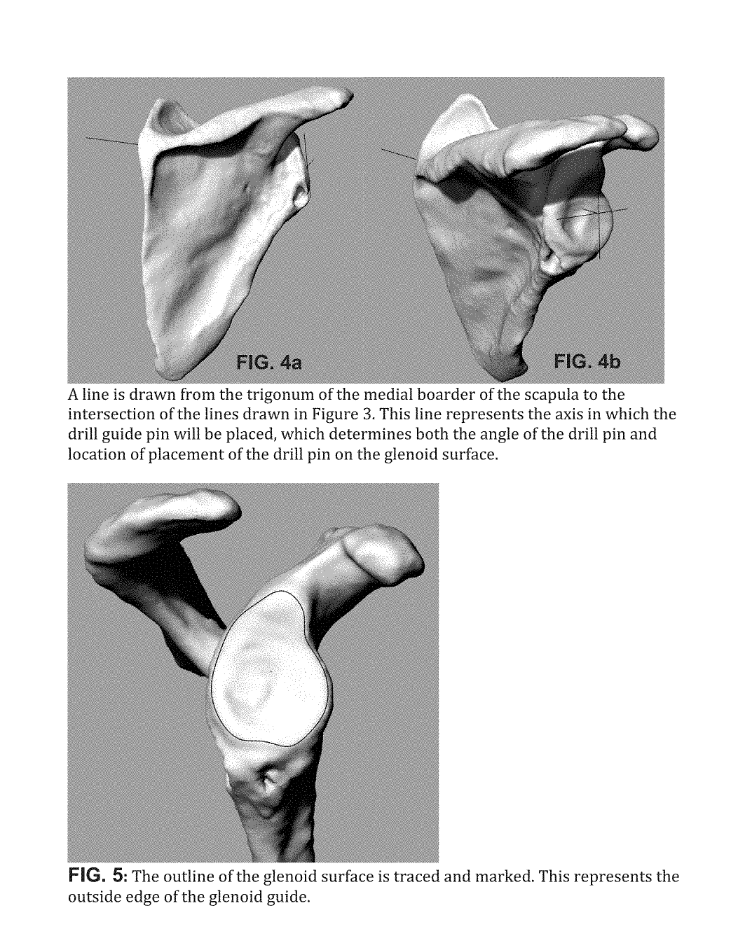

Once the virtual solid object is created, an orthogonal (en face) view of the glenoid is established and the center of the glenoid is identified by placing two perpendicular intersecting lines on the glenoid, with each line going from the widest and tallest portions of the glenoid respectively. FIG. 3a shows the en face view of the glenoid. FIG. 3b shows the horizontal and vertical lines intersecting on the glenoid face. This center point may be adjusted as needed based on glenoid deformity.

Referring to FIGS. 4a and 4b, a line is then drawn from the trigonum of the scapula to the point of intersection of the lines drawn on the glenoid en face view in the previous step. This line represents the axis in which the drill guide pin will be placed and determines both the angle of the drill pin and location of placement of the drill pin on the glenoid surface, with the angle of the drill pin orthogonal to the planned version of the final glenoid surface. Again, this line may be adjusted based on pre-operative planning and glenoid deformity to accomplish a reference point for the final guide.

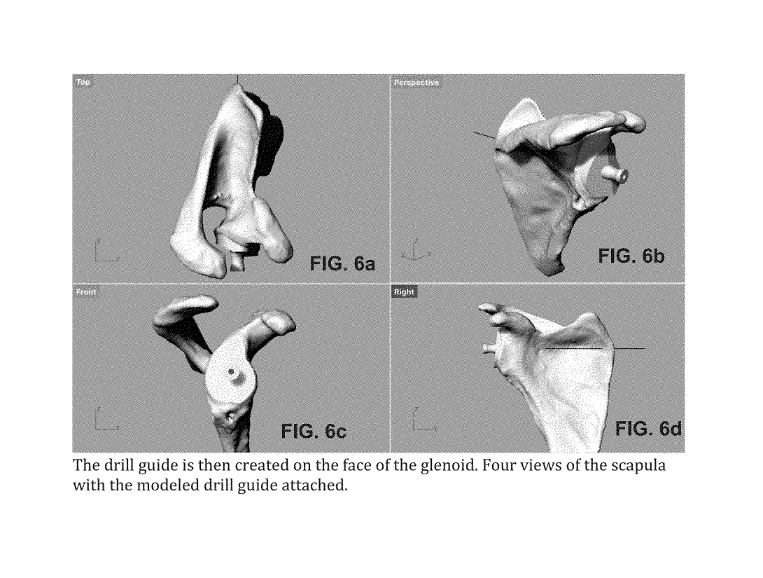

Next, the outline of the glenoid is traced and marked, as depicted in FIG. 5. This outline represents the outside edge of the glenoid guide being created. The guide is modeled based off the following: the surface of the glenoid, the line representing the axis of the drill pin, and the tracing of the glenoid surface. FIGS. 6a-d show the virtual solid object with the guide added to the glenoid face, from 4 different views. The glenoid guide may encompass the full surface of the glenoid bone, a portion of the glenoid bone or specific reference points on the glenoid as needed depending on desired guide configuration.

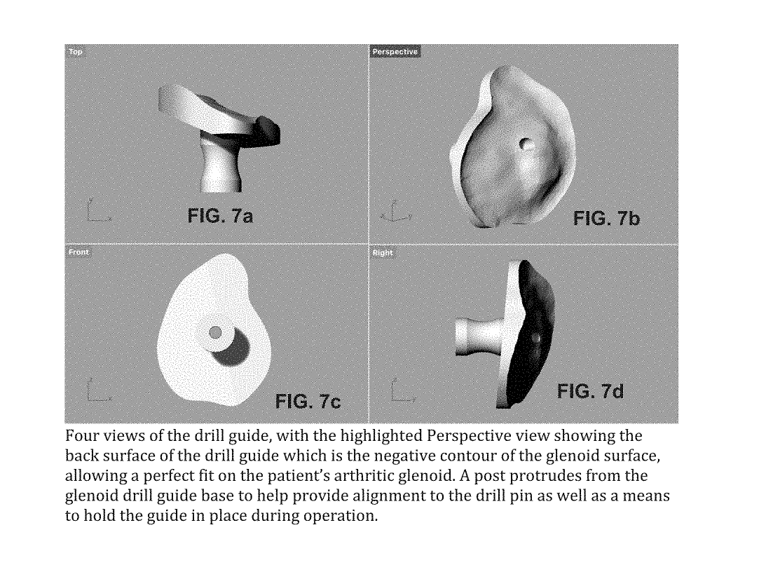

FIGS. 7a-7d are corresponding views of a virtual guide having had the surface of the glenoid subtracted from the drill guide. Note that the backside of the drill guide is the exact negative surface of the patient's glenoid surface. This allows perfect alignment of the drill guide on the glenoid and perfect placement of preoperatively planned drill pin. In addition, a post is added to the drill guide, which helps align the pin in the guide as well as provide a surface to hold the guide on the glenoid during use. Again variations of the guide are possible as needed as long as the guide can be reproducibly placed on the native glenoid bone during surgery.

Having a digital representation of the guide, as shown in FIGS. 7a-7d, the file is then saved as an STL file format. The STL file is then converted such that it can be read by 3d printers. For example, the STL file could be converted to gcode in open-sourced software. The gcode is a file type that can be read by 3D printers and dictates how the device will print the part, layer by layer. The drill guide is then printed using an FDA-approved medium, such as nylon filament, which can be sterilized for surgical use such as by means of an autoclave.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

We claim:

1. A method of creating a patient-specific surgical tool guide comprising:
scanning a target location on a patient for which a tool guide is desired;
using data from said scanning to create a virtual solid object;
identifying a surface on said image with which to mate said tool guide;
creating a virtual tool guide against said surface; and,
creating a physical tool guide from said virtual tool guide.

2. The method of claim 1 wherein scanning a target location on a patient for which a tool guide is desired comprises scanning the patient using a CT imager.

3. The method of claim 1 wherein using data from said scanning to create a virtual solid object comprises:
segmenting areas of interest of the target location;
subtracting areas not of interest from the data;
rendering the data into a 3D object;
saving said 3D object as a stereolithography (STL) file; and,
using a 3D modeling program to convert said STL file into said virtual solid object.

4. The method of claim 3 wherein segmenting areas of interest of the target location comprises filling in gaps in said data, said gaps occurring between scans of said CT scanning.

5. The method of claim 1 further comprising rotating said virtual object to achieve an orthogonal view of said surface.

6. The method of claim 5 further comprising establishing a center of said surface on said orthogonal view.

7. The method of claim 6 wherein establishing a center of said surface comprises placing two perpendicular intersecting lines on said surface, each line located to span a widest and a tallest dimension of said surface, respectively.

8. The method of claim 7 further comprising creating a third line through an intersection of said two perpendicular lines, said third line representing an axis along which a tool will be inserted into the guide.

9. The method of claim 8 wherein said third line is perpendicular to said two intersecting lines.

10. The method of claim 1 wherein creating a physical tool guide comprises 3D printing an object using from a file of the virtual tool guide.

11. A method of using a tool to modify a bone of a patient comprising:
creating a tool guide having a mating surface that mates with a target surface of a bone of a patient, said mating surface is created using from a virtual model of said target surface;
inserting a tool through an opening in said tool guide that aligns said tool with said target surface; and,
using said tool to modify said bone.

12. The method of claim 11 wherein creating a tool guide comprises:
scanning a target location containing the target surface;
creating said virtual model;
identifying said target surface representation on said virtual solid object;
creating a virtual tool guide against said surface; and,
creating said tool guide from said virtual tool guide.

13. The method of claim 12 wherein scanning the target location comprises using a CT scanner to scan the target location.

14. The method of claim 12 wherein creating said virtual model comprises:
segmenting areas of interest of the target location;
subtracting areas not of interest from the data;
rendering the data into a 3D object;
saving said 3D object as a stereolithography (STL) file; and,
using a 3D modeling program to convert said STL file into said virtual solid object.

15. The method of claim 12 wherein creating a tool guide comprises:
establishing a center of said surface comprises placing two perpendicular intersecting lines on said surface, each line located to span a widest and a tallest dimension of said surface, respectively;
creating a third line through an intersection of said two perpendicular lines, said third line representing an axis along which a tool will be inserted into the guide;
extending said third line through said virtual tool guide; and,
using said third line to form a passage through said virtual tool guide sized to accommodate said tool, said passage forming said opening in said tool guide when said virtual tool guide is used to create said tool guide.

16. A tool guide for use in modifying a bone of a patient comprising:
a surface shaped to mate with a target surface of a bone of a patient; and,
an opening sized to accommodate a tool and leading to said target surface.

17. The tool guide of claim 16 comprising nylon filament.

18. The tool guide of claim 17 wherein said nylon filament is 3D printed to form said tool guide.

19. The tool guide of claim 16 wherein said target surface is modeled from a virtual model of said bone.

20. The tool guide of claim 16 wherein said tool guide is 3D printed from a virtual tool guide.
